(19) United States
(12) Patent Application Publication
(10) Pub. No.: US 2021/0270132 A1
(43) Pub. Date: Sep. 2, 2021

(54) IN-LINE MUD LOGGING METHOD

(71) Applicant: SHELL OIL COMPANY, HOUSTON, TX (US)

(72) Inventors: Anton NIKITIN, Houston, TX (US); Amie Marie HOWS, Houston, TX (US); Ronny HOFMANN, Houston, TX (US)

(21) Appl. No.: 17/252,645

(22) PCT Filed: Jun. 18, 2019
(86) PCT No.: PCT/US2019/037649

§ 371 Date: Dec. 15, 2020

Related U.S. Application Data
(60) Provisional application No. 62/687,459, filed on Jun. 20, 2018.

(51) Int. Cl. E21B 49/00; G01N 33/28; G01T 1/36

(52) U.S. Cl. CPC E21B 49/005; G01T 1/36; G01N 33/2823

(57) ABSTRACT

A method for real-time or near real-time analysis of drilling fluid and/or drill cuttings from a well is applied to the material conveyed from the well to a mud circulation system during a drilling operation. A gamma-ray detector is provided in-line with the mud circulation system. Solids are concentered in a concentrator proximate to the gamma-ray detector. Gamma-ray spectra are detected for gamma-rays emitted from the concentrated solids. The material is removed from the concentrator. Information regarding the gamma-ray spectra representing a time correlated to drilling depth is communicated to a control unit remote from the mud circulation system.

### FIELD OF THE INVENTION

The present invention relates to mud logging and, in particular, to an in-line mud logging method for improved sampling and real- or near real-time analysis of drill cuttings.

### BACKGROUND OF THE INVENTION

In drilling operations for extracting hydrocarbons from a subsurface formation, drilling mud and/or drilling fluid (hereinafter “drilling fluid” will refer to drilling mud and/or other drilling fluids) is typically pumped downhole to carry drill cuttings to the surface. The drill cuttings, circulating drilling fluid and mud gas can provide useful information regarding strata in the subsurface formation, including, for example, without limitation, lithological type of a formation, the quality of the rock, mapping of a formation, likelihood of missed or new hydrocarbon layers or reservoirs, and the like.

Mud logging is one of the oldest methods for collecting subsurface information. Conventionally, drill cuttings carried to the surface are separated from the circulation fluid and fines with a shaker.

Typically, so-called mud loggers are on-site at the drilling operation for manually collecting a sample of drill cuttings from a conveyor, such as a shaker. In a conventional operation, a catcher board is placed at the base of the conveyor to catch drill cuttings as they fall from the end of the conveyor. After the desired sampling time, the mud logger scrapes the cuttings off the catcher board into a sample container and labels the samples for later analysis, off-site or on-site at a location remote from the conveyor.

Concentrations of naturally occurring radioactive isotopes such as K, U and Th are widely used in formation evaluation. Currently these parameters of formation are measured downhole by gamma-ray wireline tools, for example Natural Gamma-ray Spectroscopy (NGS) wireline tools, and/or measured on cuttings or core samples in the lab using X-ray Fluorescent Spectroscopy (XRF). In the case of wireline tools, substantial additional costs are imposed due to rig time required for this operation. For XRF measurements, there are limitations on the number of sample measurements a mud logger can physically process and characterize on-site.

If information delivered by mud logging is intended for the quantitative description of the subsurface, drill cutting samples should be acquired every 5 feet (1.5 m) along a well borehole. At a typical rate of penetration (ROP) of 100 ft/hr (30 m/hr), a sample of drill cuttings material would need to be acquired every 3 minutes. However, this rate of drill cutting sample acquisition is not always feasible. Beyond the physical demands of a sample acquisition every 3 minutes, on-site analysis becomes challenging due to a limited throughput of lab XRF equipment. Furthermore, there are safety concerns for people performing such sample catching operation. Accordingly, it would be advantageous to reduce or eliminate the need for a person taking manual samples, while improving a sample acquisition rate.

In an effort to address the need for drill cutting sampling and analysis of sampled cuttings material, U.S. Pat. No. 6,386,026B1 (Zamfes) describes analyzing a continuous stream of drill cuttings by conveying the cuttings from a shaker to a trough having a helical screw for moving the cuttings. A metering device holds a sample between vanes and releases one portion of the sample to a cotton bag and passes another portion to another screw conveyor to a transparent cylinder for quantitative analysis. In another approach, Zamfes (US2005/0082468A1, US2008/0202811A1) contemplates modifying the device of U.S. Pat. No. 6,386,026B1 with gamma radiation, beta radiation and sonic sensors provided on the side of an auger transporting drill cuttings. It is unclear from these later publications, but it appears that Zamfes intended the auger with sensors would replace the transparent cylinder described in U.S. Pat. No. 6,2386,026B1.

U.S. Pat. No. 7,730,795B2 (Rieberer) relates to a drill cuttings sampler having a frame for mounting on the end of a conventional vibratory shaker for supporting a perforated plate in the outflow path of drill cuttings exiting the shaker. A removable cutting collector is mounted beneath the perforated plate for allowing cuttings of a desired size to pass through to the collector, while larger cuttings are rejected and pass over the plate by washing with spray nozzles and vibration from the vibratory shaker. Samples of a pre-determined drilling interval are removed from the cutting collector for remote analysis.

EP3156587A1 (Schlumberger) describes an automated device for analyzing drill cuttings. A sampler located at the end of a shaker has at least one conveyor for transporting a sample in a canister from an outlet of a shaker to a treatment unit. A robotic manipulator takes the canister from the conveyor to a washing station and an archiving station for tagging and storing the sample.

WO2012/100283A1 (Technological Resources Pty) relates to a sampling and analysis system for coupling with a drilling rig. A frame supports a drill cuttings handling system and an elemental analysis device and is coupled to a drilling rig platform with hydraulic rams. Drill cuttings are transported from a conveyor near the hole being drilled to a sampler splitter to produce two or more sample streams. One stream is directed to a feed chute and then to another conveyor in the analyzer, through a levelling device. Once this portion of the sample is transported past the analysis zone of the analyzer, it falls off the downstream end to the ground. The analyzer and conveyor and related equipment, are supported on a vibration isolation system.

Deficiencies of the systems described above include not allowing for real-time or near real-time analysis useful for more efficient decision-making, requiring modifications to drilling operation equipment, and/or they are very complex. Still another deficiency of the prior system having sensors on a transport tube is that the drill cuttings are not concentrated enough to measure a meaningful result. While it may be suitable for some applications, diluted drill cutting samples are a problem when deeper wells are drilled, where significantly more drilling fluid is required to remove the drill cuttings from deeper depths. There is a need for a simple method that can be implemented in an existing drilling operation without undue modification. There is also a need for an in-line method that can concentrate drill cuttings for a more meaningful result.

### SUMMARY OF THE INVENTION

According to one aspect of the present invention, there is provided a method for real-time or near real-time analysis of material selected from drilling fluid, drill cuttings and combinations thereof from a well, the material, having at least a solids content and a liquid content, conveyed from the well to a mud circulation system during a drilling operation, the method comprising: providing a gamma-ray detector in-line with the mud circulation system; concentrating a portion of the solids content from the material in a concentrator proximate to the gamma-ray detector, the portion of the solids content representing a time correlated to a drilling depth; detecting gamma-ray spectra using the gamma-ray detector for the concentrated solids content emitting gamma-rays; removing the concentrated solids content from the concentrator; and communicating information regarding the gamma-ray spectra to a control unit remote from the concentrator.

### BRIEF DESCRIPTION OF THE DRAWINGS

The method of the present invention will be better understood by referring to the following detailed description of preferred embodiments and the drawings referenced therein, in which:

FIG. 1 is a side elevation view illustrating the flow of material from a well to a mud circulation system illustrating one embodiment of a concentrator and a gamma-ray detector of the present invention for real-time or near real-time analysis of material from the well (for better showing particular features of the invention, the drawing is not necessarily to scale);

FIG. 2 is a side elevation view illustrating the flow of material from a well to a mud circulation system illustrating another embodiment of a concentrator and a gamma-ray detector of the present invention for real-time or near real-time analysis of material from the well (for better showing particular features of the invention, the drawing is not necessarily to scale);

FIG. 3 is a front perspective view of one embodiment of an in-line gamma-ray detection apparatus suitable for use in the method of the present invention, particularly for the embodiment of FIG. 1;

FIG. 4 is a front elevation view of the apparatus of FIG. 3;

FIG. 5 is a top plan view of the apparatus of FIG. 3;

FIG. 6 is a front perspective view of the analysis body of the apparatus of FIG. 3 showing a sample accumulation tray in an open position; and

FIG. 7 is a cross-sectional view of an analysis body of the apparatus of FIG. 3 along the line 7-7;

FIG. 8 is a cross-sectional view of another embodiment of an in-line gamma-ray detection apparatus suitable for use in the method of the present invention; and

FIGS. 9A and 9B are cross-sectional views of the in-line gamma-ray detection apparatus of FIG. 8 showing operation of the in-line gamma-ray detection apparatus.

### DETAILED DESCRIPTION OF THE INVENTION

The present invention provides a method for real-time or near real-time analysis of material from a well. Material transported from a well has at least a solids content and a liquid content and includes drilling fluid, drill cuttings and combinations thereof. Advantageously, the method may be applied to an existing mud circulation system.

The in-line mud logging method of the present invention advantageously reduces the need for mud-logging personnel to manually collect samples, for example, from a conventional catcher board. Moreover, samples can be taken at a higher frequency, thereby providing greater vertical resolution of a well.

Because drill cuttings comprise pieces of the formation crushed by the drill bit during a drilling operation, properties of these cuttings are representative of the properties of the formation from which the cuttings originated. As such, if traced back to a wellbore depth, these cuttings may be sampled and analyzed to provide information about the formation properties present at that depth within the wellbore.

The formation properties determined by the method of the present invention provide information to assess the ability of a formation to produce hydrocarbons and/or to estimate the mechanical properties of the formation, for example, to determine frackability of the formation.

In accordance with the present invention, a gamma-ray detector is provided in-line with the mud circulation system. A concentrator is disposed proximate to the gamma-ray detector and is adapted to concentrate a portion of the solids content.

Gamma-ray emissions from the concentrated solids content are detected by the gamma-ray detector. After the gamma-rays are detected, the concentrated solids content is removed from the concentrator. Information regarding the gamma-ray spectra provided by the gamma-ray detector is communicated to a control unit that is remote from the mud circulation system.

Preferably, the weight of the material in the concentrator is measured, for example, with a weight sensor. More preferably, concentrated solids content is removed from the concentrator once the measured weight reaches a predetermined weight of material. Alternatively, concentrated solids content is removed from the concentrator once a substantially constant weight of material is measured. As a further alternative, concentrated solids content is removed at a desired time interval.

Given the typical rate of drill cuttings produced and transported in a typical drilling operation, the concentrator may reach the desired sample size every 20 to 30 seconds. Gamma-ray spectra can then be measured every several seconds to ensure a more accurate weight normalization of the resulting spectrum accumulated for each sample of concentrated solids content.

Preferably, the gamma-ray spectra are corrected for any background source of gamma-rays. A background spectrum is preferably measured in situ when there is no drilling and, as a result, when no drill cuttings are produced. Preferably, the gamma-ray spectra are normalized by the control unit for the sample weight measured during the detection time.

Also, after normalization, an average gamma-ray spectrum may be calculated for gamma-ray spectra acquired between consecutive samples. The average gamma-ray spectrum corresponds to the gamma-ray spectrum of the formation averaged over the depth interval of interest. The decomposition of the average gamma-ray spectrum into K, U and Th contributions provides information about averaged concentrations of K, U and Th for the formation across the depth interval of interest.

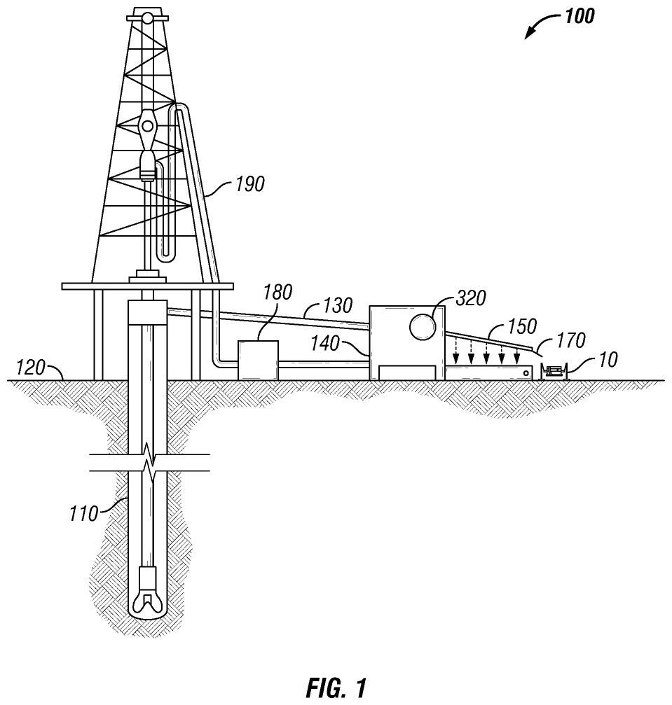

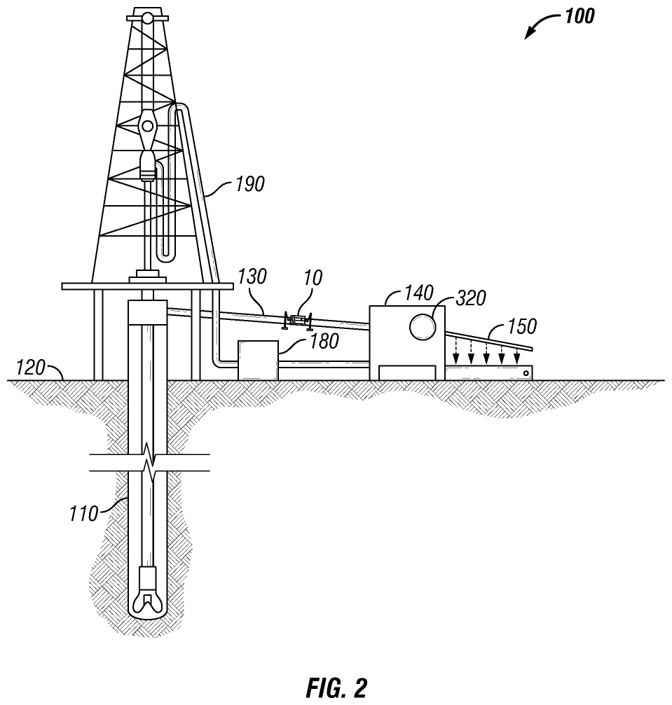

Two embodiments of a mud circulation system 100 of the present invention are depicted in FIGS. 1 and 2. Drill cuttings are transported from a well 110 to the surface 120 with drilling fluid. A mud pipeline 130 conveys material from the well 110 to a possum belly 140 which allows any entrained gasses to be released. The possum belly 140 also reduces the mud flow rate before the drilling fluid is transported to a conveyor 150. The conveyor 150 may be a shaker conveyor, a vibratory conveyor or a combination thereof. A proximate end of the conveyor 150 receives material, via the mud pipeline 130 and the possum belly 140, from the well 110. Preferably, the conveyor 150 acts to separate a majority of the drilling fluid from the drill cuttings, as depicted by the downward arrows from the conveyor 150. The drilling cuttings are separated from the drilling fluid and the drilling fluid is returned downhole, with or without further treatment, to transport more drill cuttings to the surface 120.

In the embodiments of FIGS. 1 and 2, the drilling fluid separated from drill cuttings is returned to the well 110 using a mud pump 180 and tubing 190. Drilling fluid can be exposed to the other treatments after being cleaned at the conveyor 150 before returning to the well bore, for example, without limitation, treatment in a settling tank and the like.

An in-line gamma-ray apparatus 160 is disposed in the path of material transported from the well. In the embodiment of FIG. 1, the gamma-ray detection apparatus 160 is disposed at the distal end of the conveyor 150. In the embodiment of FIG. 2, the gamma-ray detection apparatus 160 is disposed at the mud pipeline 130.

In a preferred embodiment, the concentrator and the gamma-ray detector are integrated into a single apparatus. More preferably, the apparatus is an embodiment described in co-pending application entitled “In-line Mud Logging Apparatus” filed in the USPTO on the same day as the present application, as a provisional application, the entirety of which is incorporated by reference herein.

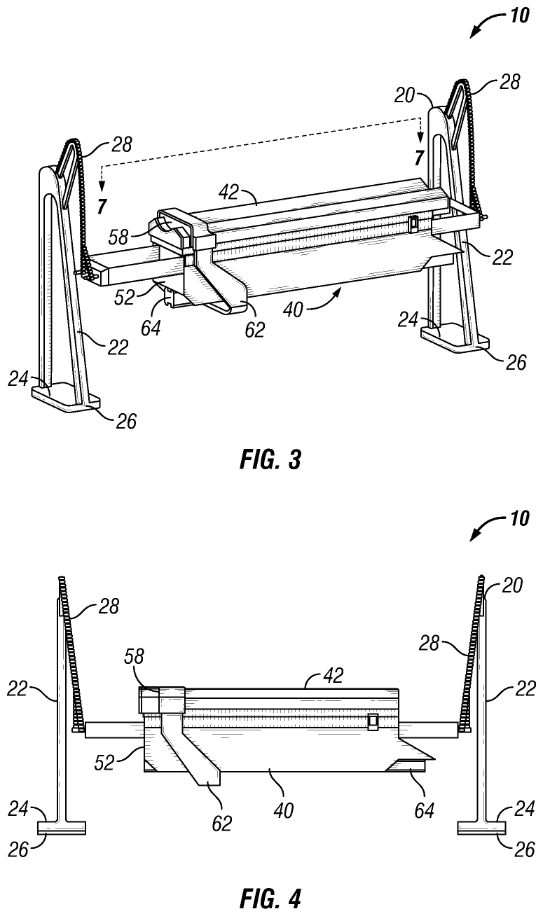

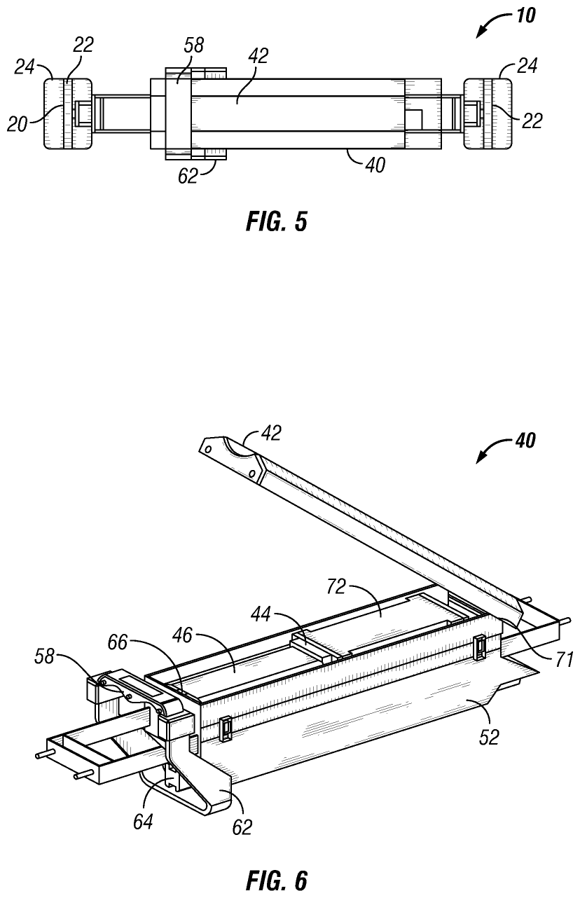

A particularly preferred embodiment of the apparatus for use in the method of the present invention is depicted in FIGS. 3-7. An in-line mud logging apparatus 10 has a mounting frame 20 and an analysis body 40. The in-line mud logging apparatus 10 is adapted to be in material communication with the conveyor 150 of a conventional mud circulation system, for example, the embodiment of FIG. 1. By “material communication,” we mean that the in-line mud logging apparatus 10 receives material directly from the conveyor 150. Preferably, the apparatus 10 receives material from the conveyor 150 without being directly attached to the conveyor 150. Conventionally, material falls from the conveyor for disposal and/or further treatment. Preferably, material falls from a distal end of the conveyor 150 onto the in-line mud logging apparatus 10. In this embodiment, the apparatus 10 is deployed in a location typically reserved for a conventional catcher board, so that little to no retrofit of the mud circulation system 100 is required.

Optionally, as shown in FIG. 1, a funnel 170 is provided on or near the distal end of the conveyor 150 for selectively channeling all or a portion of the material from the conveyor 150 to the in-line mud logging apparatus 10.

In the FIG. 1 embodiment, a preferred embodiment of the in-line mud logging apparatus 10 is provided with the mounting frame 20 to vibrationally isolate the analysis body 40 from vibrations in the mud circulation system 100 and surrounding equipment. As shown in FIG. 3, in one embodiment, the mounting frame 20 is a two-part structure with two supporting legs 22 that are not directly connected to one another. Alternatively, the supporting legs 22 may be connected to one another with crossbar (not shown). The supporting legs 22 have a base 24 for supporting the analysis body 40 on the ground, a platform or other surface. Preferably, the base 24 is provided with a vibration-damping material 26. A particularly suitable vibration-damping material 26 is a rubber material, for example, natural, synthetic or recycled rubber, a rubber composite or a rubber laminate. However, other materials for suitable for vibration-damping will be apparent to those skilled in the art. Preferably, the vibration-damping material 26 can suppress or damp higher frequency vibrations.

In a preferred embodiment, the analysis body 40 is suspended from the mounting frame 20 with chains 28. The chains 28 advantageously suppress or damp lower frequency vibrations.

In the embodiment of FIG. 1, the concentrator for concentrating a portion of the solids content from the material conveyed from the well is a sample accumulation tray 42. Referring to

FIGS. 3-7, the analysis body 40 has the sample accumulation tray 42 to collect material falling from the conveyor. Because the cuttings are wet, at least a portion of the material tends to fall directly onto the sample accumulation tray 42 as opposed to falling forward of the sample accumulation tray 42. It has been observed that the material falling from the conveyor 150 will fall onto any material in the sample accumulation tray 42 until a substantially steady state is reached where any new material rolls off the material already in place or displaces some material previously accumulated in the sample accumulation tray 42. Accordingly, the sample obtained is generally a representative sample. Moreover, the sample is a concentrated sampling of a drilling interval, allowing for more meaningful results.

In the embodiment shown in FIGS. 3-7, the sample accumulation tray 42 acts as a cover for the interior of the analysis body 40, which houses a gamma-ray detector 44, preferably a gamma-ray spectrometer.

Natural Gamma-ray Spectroscopy (NGS) analysis of drill cuttings provides information about the type of rock being drilled. Such information is useful for drilling decisions. Potassium (K), thorium (Th), and uranium (U) are three natural sources of gamma-ray radiation present in the earth. Each of these elements emit gamma-rays with known energies, which are unique to the particular element. Shales can be distinguished from other types of rock due to the relatively high levels of these gamma-ray radiating elements present in shale. Accordingly, the presence of shale is useful information for making decisions about a drilling operation.

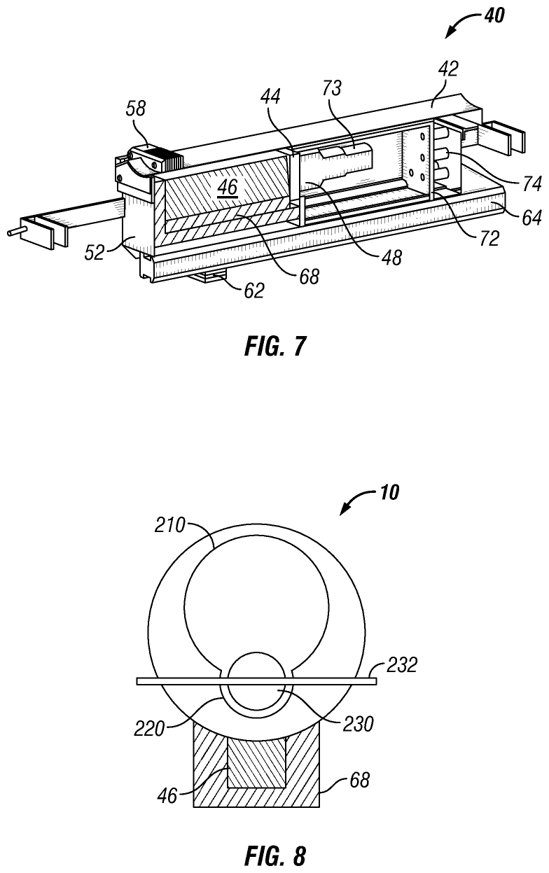

FIG. 6 illustrates the sample accumulation tray 42 in an open position to reveal the gamma-ray detector 44, while FIG. 7 is a cross-sectional view of the analysis body 40. The gamma-ray detector 44 preferably comprises a gamma-ray detecting scintillation crystal 46, which is preferably optically coupled to a photomultiplier 48 for detecting photons emitted by the scintillation crystal 46. Detector electronics 73 containing a high voltage power supply and a signal processor is connected to the photomultiplier. Other electronic components required for NGS measurements will be understood by those skilled in the art of, for example, NGS wireline tools.

Preferably, the scintillation crystal 46 has a length extending along the axis of the analysis body 40 for capturing as much of the gamma-ray signal from the sample in the tray as possible. The scintillation crystal 46 is formed of a solid inorganic luminescent material that generates photons of light in response to contact with gamma-rays. Such inorganic luminescent materials include, for example, without limitation, sodium iodide (NaI), cesium iodide (CsI), and bismuth germanate (Bi₄Ge₃O₁₂). Sodium iodide is a particularly preferred solid inorganic luminescent material for use in the in-line mud logging apparatus 10 of the present invention because relatively large sodium iodide crystals may be formed easily and economically.

The inorganic luminescent material may include one or more activators to enhance emission of photons by the scintillation crystal 46 that are within a range of wavelengths that are detectable by the photomultiplier 48. Such activators may be present as impurities in the scintillation crystal material, and may be introduced to the crystal as a dopant. Thallium is a preferred activator for use in a sodium iodide or cesium iodide scintillation crystal. A thallium-doped sodium iodide crystal is a preferred inorganic scintillation crystal material for use in the gamma-ray detector 44.

The photomultiplier 48 may be any conventional photomultiplier. As noted above, the photomultiplier 48 is optically coupled to the scintillation crystal 46, and may be physically coupled to the scintillation crystal 46 by locating an end of the scintillation crystal 46 in a receiving portion of the photomultiplier 48. Optical coupling grease, for example a silicon grease, may be applied at a contact interface between the end of the scintillation crystal 46 and the receiving portion of the photomultiplier 48 to reduce the loss of scintillation photons by preventing reflection of the photons at the contact interface. The photomultiplier 48 generates an electrical signal from detected photons of light emitted by the scintillation crystal 46 that is proportional to the gamma-ray energy absorbed in the scintillation crystal 46. The electrical signal produced by the photomultiplier 48 may be used to generate a gamma-ray spectrum for analysis.

The base 52 of the analysis body 40 houses the scintillation crystal 46. Preferably, the sample accumulation tray 42 covers the base 52 in such a way as to reduce penetration of fluids associated with the drill cuttings or weather into the base 52. In a preferred embodiment, the sample accumulation tray 42 is attached to the base 52 with a hinge 71, thereby allowing the opposing end of the sample accumulation tray 42 to move in response to weight for communicating with a weight sensor 66. This and other connections that allow for movement of the sample accumulation tray 42 in response to weight allows the weight sensor 66 to quantify the weight in the sample accumulation tray 42.

In one embodiment, the gamma ray detector 44 is coupled to the electronics enclosure 78 which encloses detector photomultiplier 48, detector electronics 47 and any other electronic components required to operate the analysis body including communication module, weight sensor electronics, control module of an actuator 64 that moves wiper 58, power supplies, electric batteries supplying power, and the like. To reduce hazardous and potentially explosive interaction of hydrocarbon vapors present at the drilling rig with electronic components, the electronic enclosure 78 is sealed, preferably hermetically, and is filled with an electrically insulating fluid (not shown). An example of a suitable electrically insulating fluid is FLUORINERT™ available from 3M. Preferably, electronic components within the electronic enclosure 72 are connected with insulated, more preferably hermetically isolated, electrical connectors 74.

In another embodiment, the electronic enclosure 72 is coupled, preferably hermetically, to the gamma ray detector 44 and is filled with a gas at a pressure slightly higher than atmospheric pressure. Such positive pressure difference inside of electronics enclosure 72 reduces penetration of any hydrocarbon vapors in the environment surrounding the analysis body 40, thereby reducing the chance of explosion. This positive pressure difference can be maintained using an external gas reservoir (not shown) equipped with pressure control equipment and connected to electronics enclosure 72 by a gas line (not shown) as will be understood by those skilled in the art of electronics equipment isolation from hazardous and explosive substances.

The base 52 has a gamma ray detector shield 68. The detector shield 68 surrounds the gamma-ray detector 44 in such a way as to maximize suppression of gamma-ray signals emitted by any gamma-ray sources in the area of deployment, other than cuttings samples in the sample accumulation tray 42. The detector shield 68 is formed of a material having a high efficiency of gamma-ray absorption effective to reduce detection of any gamma-ray radiation beyond the gamma-rays emitted from the desired sample. In one embodiment, the material is lead. Alternatively, the detector shield 68 can be made of tungsten, W-Cu alloy, mercury or any other high-density material as it can be understood by those skilled in the art of radiation shielding design.

As shown in the embodiments depicted in FIGS. 3-7, the sample accumulation tray 42 has a shape for holding a pre-determined sample size. Material falling from the conveyor falls into the sample accumulation tray 42 so that the gamma-ray detector 44 can detect gamma-ray emission from the material. The sample accumulation tray 42 allows the sample to be concentrated to produce a more meaningful result. After the gamma-rays are detected, the accumulated material is removed from the sample accumulation tray 42. Information about the gamma-ray spectra provided by the gamma-ray detector 44 is communicated to a control unit (not shown) that is remote from the distal end of the conveyor 150. Information is preferably communicated via a communication module (not shown) located inside the electronics enclosure 72.

Preferably, the weight of the material in the sample accumulation tray 42 is measured, for example, with a weight sensor 66. More preferably, accumulated material is removed from the sample accumulation tray 42 once the measured weight reaches a predetermined weight of material. Alternatively, accumulated material is removed from the sample accumulation tray 42 once a substantially constant weight of material is measured. It has been observed that the material falling from the conveyor 150 will fall onto any material in the sample accumulation tray 42 until a substantially steady state is reached where any new material rolls off the material already in place or displaces some material previously accumulated in the sample accumulation tray 42. As a further alternative, accumulated material is removed at a desired time interval.

When a desired sample size is accumulated in the sample accumulation tray 42, which is detected by the weight sensor 66, the sample is removed from the sample accumulation tray 42 by a sample removal device. A preferred device for removal is a wiper 58. In a preferred embodiment illustrated in FIGS. 3-7, the wiper 58 is carried by a yoke 62 that is adapted to travel along an actuator 64 along the length of the analysis body 40. The wiper 58 is preferably formed of a flexibly resilient material to conform with the shape of the sample accumulation tray 42, while being rigid enough to sweep or push the sample out of the sample accumulation tray 42. Preferably, the wiper 58 is formed of rubber, plastic or a combination thereof.

In a preferred embodiment illustrated in FIGS. 3-7, the actuator 64 is a pneumatic cylinder equipped with the two end switches and a valve controlled by the control module (not shown) located inside of the electronics enclosure 72 and connected to the external source of the pressurized gas (not shown) required for the actuator movement as it could be understood by those skilled in the art of pneumatic motion system design. The wiper 58 is connected by the yoke 62 to the actuator 64 and as a result the actuator 64 moves the wiper 58 along the length of the analysis body 40 from one side of the sample accumulation tray 42.

In the embodiment of FIGS. 3-7, the sample accumulation tray 42 has two free ends to allow movement of the wiper 58 along the length of the sample accumulation tray 42. Preferably, the yoke 62 allows the wiper 58 to travel past the free ends of the sample accumulation tray 42, thereby reducing any inadvertent trapping of sample between the wiper 58 and the sample accumulation tray 42.

The desired sample size is preferably measured with weight sensor 66, for example, as shown in FIG. 6. Other locations and types of weight sensors 66 may be used within the scope of the present invention.

In order to provide a quantitative analysis of the elements detected by the gamma-ray detector 44, weight information of the sample is also communicated via the communication module located inside the electronics enclosure 72 to the control unit so that the concentration of elements detected by the gamma-ray detector 44 can be quantified on a weight basis.

Preferably, the yoke 62 is actuated to move along the actuator 64 when the material in the sample accumulation tray 42 reaches a predetermined weight that is sensed by the weight sensor 66. In another preferred embodiment, the yoke 62 is actuated when the weight sensor 66 detects that the weight of material in the sample accumulation tray 42 is substantially constant. Alternatively, the yoke 62 may be actuated by a time interval or by external command.

As an alternative to a wiper 58, a sample can be removed from the sample accumulation tray 42 by rotating the sample accumulation tray 42, pressure-washing, or other means within the scope of the present invention.

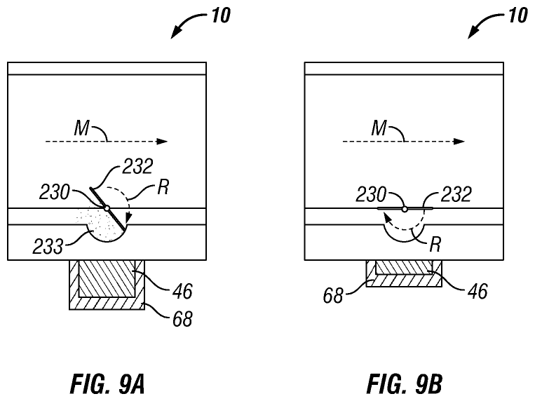

In the embodiment of FIG. 2, the concentrator for concentrating a portion of the solids content from the material conveyed from the well is a trap 220. FIGS. 8, 9A and 9B illustrate a suitable embodiment of the in-line gamma-ray detection apparatus 10. The pipe insert 210 has a trap 220 cut in a base of the pipe insert 210. A rotating trap lid 230 is rotatably mounted on rotating pin 232. As illustrated more clearly in FIGS. 9A and 9B, the trap lid 230 is rotated about rotating pin 232 to swing the trap lid 230 into the flow of material, depicted by arrow M. In this orientation, the trap lid 230 is rotated in a clockwise direction, depicted by arrow R. When the trap lid 230 is in the flow M, solids are concentrated in the trap 220 (see FIG. 9A). When the trap lid 230 is rotated out of the flow M, the material flow removes any solids concentrated in the trap 220 (see FIG. 9B).

A gamma-ray detector 44 capable of measuring spectrum of the detected gamma-ray signal preferably comprises a gamma-ray detecting scintillation crystal 46, which is preferably optically coupled to a photomultiplier (not shown) for detecting photons emitted by the scintillation crystal 46. The gamma-ray detector 44 is surrounded by the gamma -ray detector shield 68 made of the dense material, preferably lead. The detector shield 68 absorbs gamma-rays emitted by any gamma-ray sources present outside of the pipe insert 210, such as soil etc. The gamma-ray detector shield 68 reduces any contribution of gamma-ray radiation beyond the gamma-rays emitted from the desired sample.

Referring now to FIGS. 9A and 9B, in operation, the trap lid 230 is rotated into the flow of material, M, to concentrate solids 233 in the material. Gamma ray detector 44 acquires spectrum of the gamma-ray signal every several seconds. The amount of solid concentrated in the trap 220 is estimated by calculation and/or derived from measurements. Calculations may be based on known mud flow rate, rate of the penetration, trap shape or volume and other parameters. Alternatively, or in combination, measurements of concentrated solids volume using an ultra-sound sensor (not shown), measurements of concentrated solids weight using a weight sensor (not shown), and/or measurements by gamma ray densitometer (not shown), may be used. The information about the amount of concentrated solids in the trap is used to normalize gamma-ray spectra acquired by the gamma-ray detector 44 to convert the normalized spectra into concentration of U, K and Th in the solids. Preferably, the normalized gamma-ray spectra are corrected for any background source of gamma-rays. When the amount of concentrated solids in the trap 220 reaches some threshold level or after a defined time interval the trap lid 230 is rotated (see FIG. 9B) to allow the material flow M to remove the concentrated solids 233 from the trap 220. After the trap 220 is cleaned by the material flow, for example, based on sensor readings or after a predefined time interval, the trap lid 230 is rotated into the material flow M to again concentrate solids.

The in-line mud logging method of the present invention may be used, for example, for the system described in co-pending application entitled “In-line Mud Logging System” filed in the USPTO on the same day as the present application, as a provisional application, the entirety of which is incorporated by reference herein.

While preferred embodiments of the present invention have been described, it should be understood that various changes, adaptations and modifications can be made therein within the scope of the invention(s) as claimed below.

1. A method for real-time or near real-time analysis of material selected from drilling fluid, drill cuttings and combinations thereof from a well, the material, having at least a solids content and a liquid content, conveyed from the well to a mud circulation system during a drilling operation, the method comprising:
providing a gamma-ray detector in-line with the mud circulation system;
concentrating a portion of the solids content from the material in a concentrator proximate to the gamma-ray detector, the portion of the solids content representing a time correlated to a drilling depth;
detecting gamma-ray spectra using the gamma-ray detector for the concentrated solids content emitting gamma-rays;
removing the concentrated solids content from the concentrator; and
communicating information regarding the gamma-ray spectra to a control unit remote from the mud circulation system.

2. The method of claim 1, further comprising the step of measuring a weight of the concentrated solids content in the concentrator.

3. The method of claim 2, wherein the concentrated solids content is removed from the concentrator when the measured weight reaches a predetermined weight of material.

4. The method of claim 2, wherein the concentrated solids content is removed from the concentrator when the measured weight remains substantially constant.

5. The method of claim 2, wherein the concentrated solids content is removed from the concentrator at a predetermined time interval.

6. The method of claim 2, wherein the control unit further comprises the step of normalizing detected gamma-ray spectra for a concentrated solids content sample with the weight measured for the concentrated solids content sample.

7. The method of claim 6, wherein the control unit further comprises the step of calculating an average gamma-ray spectrum for a consecutive two concentrated solids content samples, thereby providing an averaged gamma-ray spectrum for the concentrated solids content for a time interval between two times correlated to drilling depth.

8. The method of claim 1, wherein the control unit further comprises the step of correcting the detected gamma-ray spectra with background gamma-ray spectra.

9. The method of claim 1, wherein the concentrated solids content emitting gamma-rays comprises an element selected from the group consisting of potassium, uranium, thorium, and combinations thereof.

10. The method of claim 1, wherein the concentrator is disposed at a distal end of a conveyor in the mud circulation system.

11. The method of claim 10, wherein the conveyor is selected from the group consisting of a shaker conveyor, a vibratory conveyor, and combinations thereof.

12. The method of claim 1, wherein the concentrator is disposed in direct communication with a possum belly in the mud circulation system.
